(19) United States
(12) Patent Application Publication
(10) Pub. No.: US 2023/0208211 A1
(43) Pub. Date: Jun. 29, 2023

(54) A METHOD AND A DEVICE FOR RECEIVER POSITION DETECTION IN MULTI-TRANSMITTER WIRELESS POWER TRANSFER SYSTEMS WITHOUT RECEIVER SENSORS

(71) Applicant: AALTO UNIVERSITY FOUNDATION SR, Aalto (FI)

(72) Inventors: Prasad Kumara Sampath JAYATHURATHNAGE, (LK); Fu LIU, (CN); Sergei TRETYAKOV, (RU)

(21) Appl. No.: 18/010,778

(22) PCT Filed: Jun. 16, 2020
(86) PCT No.: PCT/FI2020/050432

§ 371 Date: Dec. 15, 2022

(51) Int. Cl. H02J 50/90; H02J 50/60; H02J 50/10; H02J 50/40

(52) U.S. Cl. CPC H02J 50/90; H02J 50/60; H02J 50/10; H02J 50/402

(57) ABSTRACT

Various example embodiments relate to enhancing excitation and output power in multi-transmitter wireless power transfer system. Transmitter coils are arranged such that they are substantially uncoupled from each other. The transmitter coils are activated one by one to measure induced voltage across adjacent transmitter coils. The induced voltages are used to determine mutual inductance ratios of transmitter-receiver pairs to obtain current distribution coefficients indicative of the position of the receiver for optimal excitation. A device, a method, and a computer program are disclosed.

### TECHNICAL FIELD

Various example embodiments generally relate to the field of electrical engineering. In particular, some example embodiments relate to wireless power transfer with multiple transmitters.

### BACKGROUND

Wireless power transfer systems are used for transmission of electrical energy without wires as a physical link. Wireless power transfer is beneficial for many applications including consumer electronics applications, medical implants, and electric vehicle charging. Multi-transmitter wireless power transfer, where multiple power transmitters (Txs) are used to energize single or multiple receivers (Rxs), can be used to extend the mobility of the Rx, for example, in charging applications. In a multi-Tx wireless power system, detection of Rx location is important for the optimal excitation of the transmitters.

### SUMMARY

This summary is provided to introduce a selection of concepts in a simplified form that are further described below in the detailed description. This summary is neither intended to identify key features or essential features of the claimed subject matter, nor is intended to be used to limit the scope of the claimed subject matter.

Example embodiments provide a device and a method to improve output power and efficiency of a wireless power transfer system by detecting a position of a receiver without any measurements from the receiver. In an example embodiment, an efficient current distribution in Tx coils is determined by estimating mutual inductance ratios between each Tx-Rx pairs using only Tx-side measurements. These benefits may be achieved by the features of the independent claims. Further implementation forms are provided in the dependent claims, the description, and the drawings.

According to an aspect, there is provided a device comprising at least three transmitter coils, the transmitter coils arranged such that all the transmitter coils are substantially uncoupled from each other; a measuring circuitry configured to measure voltage across individual transmitter coils; a power source configured to supply power to the transmitter coils independently or simultaneously; a processor; and a memory comprising program code which, when executed by the processor, causes the device at least to activate transmitter coils one by one by the power source such that only one transmitter coil is activated at a time; measure, by the measuring circuitry, induced voltages of at least two transmitter coils adjacent to the activated transmitter coil; determine mutual inductance ratios between the adjacent transmitter coils and at least one receiver based on the induced voltages; and determine current distribution coefficients of the transmitter coils indicative of a position of the at least one receiver with respect to the transmitter coils based on the mutual inductance ratios.

In an embodiment, the device is further caused to initiate power supply to the transmitter coils according to the determined current distribution coefficients.

In an embodiment, in addition or alternatively, the transmitter coils are positioned in a planar arrangement.

In an embodiment, in addition or alternatively, the transmitter coils are positioned in a ball-shaped arrangement.

In an embodiment, in addition or alternatively, the transmitter coils are positioned in a container-shaped arrangement.

In an embodiment, in addition or alternatively, the transmitter coils are positioned such that a normalized displacement d/Rout between the adjacent transmitter coils results approximately zero coupling coefficient.

In an embodiment, in addition or alternatively, the device is integrated to a furniture or infrastructure.

In an embodiment, in addition or alternatively, the transmitter coils are activated one by one at a predetermined interval.

In an embodiment, in addition or alternatively, the device is caused to compare the mutual inductance ratios after each induced voltage measurements to detect transmitter coils associated with higher mutual inductance ratios; and activate next only the transmitter coils adjacent to the transmitter coils associated with the higher mutual inductance ratios.

In an embodiment, in addition or alternatively, the device is caused to determine that there are no receivers nearby in response to the measurements indicating that no voltage is induced to any of the transmitter coils; and enter into a sleep mode when there are no receivers nearby.

In an embodiment, in addition or alternatively, the device is caused to measure power from the active transmitter coil; detect an increase in the power from the active transmitter coil while there are no induced voltages across the adjacent transmitter coils; and determine there is a metal object near the active transmitter coil.

In an embodiment, in addition or alternatively, the device is caused to enter into the sleep mode in response to detecting the metal object near the active transmitter coil while there are no receivers nearby.

In an embodiment, in addition or alternatively, the device is caused to notify a user about the metal object.

According to a second aspect, a method is provided. The method comprises activating transmitter coils one by one by a power source such that only one transmitter coil is activated at a time, wherein the transmitter coils comprise at least three transmitter coils and all the transmitter coils are substantially uncoupled from each other; measuring induced voltages at adjacent transmitter coils of the activated transmitter coil;

determining mutual inductance ratios between the adjacent transmitter coils and a receiver based on the induced voltages; and determining current distribution coefficients of the transmitter coils indicative of a position of the receiver with respect to the transmitter coils based on the mutual inductance ratios.

In an embodiment, the method comprises initiating power supply to the transmitter coils according to the determined current distribution coefficients.

In an embodiment, in addition or alternatively, the transmitter coils are activated one by one at a predetermined interval.

In an embodiment, in addition or alternatively, the method further comprises comparing the mutual inductance ratios after each induced voltage measurements to detect transmitter coils associated with higher mutual inductance ratios; and activating next only the transmitter coils adjacent to the transmitter coils associated with the higher mutual inductance ratios.

In an embodiment, in addition or alternatively, the method comprises determining that there are no receivers nearby in response to the measurements indicating that no voltage is induced to any of the transmitter coils; and entering into a sleep mode when there are no receivers nearby.

In an embodiment, in addition or alternatively, the method comprises measuring power from the active transmitter coil; detecting an increase in the power from the active transmitter coil while there are no induced voltages across the adjacent transmitter coils; and determining there is a metal object near the active transmitter coil.

In an embodiment, in addition or alternatively, the method comprises entering into the sleep mode in response to detecting the metal object near the active transmitter coil while there are no receivers nearby.

In an embodiment, in addition or alternatively, the method comprises notifying a user about the metal object.

According to a third aspect, a computer program product is provided, comprising program code which, when executed by at least one processing unit, causes the at least one processing unit to perform the method of the second aspect.

According to a fourth aspect, a computer readable medium is provided, comprising the computer program product of the third aspect.

Many of the attendant features will be more readily appreciated as they become better understood by reference to the following detailed description considered in connection with the accompanying drawings.

### DESCRIPTION OF THE DRAWINGS

The accompanying drawings, which are included to provide a further understanding of the example embodiments and constitute a part of this specification, illustrate example embodiments and together with the description help to understand the example embodiments. In the drawings:

FIG. 1 illustrates an example of an equivalent circuit of a device comprising multiple transmitter coils for wireless power transfer to a receiver according to an embodiment.

FIG. 2 illustrates an example of coupling coefficient variation between two planar circular spiral transmitter coils with respect to a displacement between the transmitter coils according to an embodiment.

FIG. 3 illustrates an example arrangement of disc-shaped transmitter coils and possible positions of a receiver with respect to the transmitter coils according to an embodiment.

FIG. 4 illustrates an example diagram of current distribution coefficients with respect to each transmitter coil based on positions of a receiver according to an embodiment.

FIG. 5 illustrates an example diagram of theoretical and experimental results of current distribution coefficients with respect to three transmitter coils of a prototype device based on positions of a receiver according to an embodiment.

FIG. 6 illustrates an example of a block diagram of a device configured to practice one or more example embodiments according to an example embodiment.

FIG. 7 illustrates an example of a device configured for wireless power transfer when the device is integrated to a furniture according to an embodiment.

FIG. 8 illustrates an example of a method for determining a position of a receiver based on measurements from transmitter coils according to an embodiment.

Like references are used to designate like parts in the accompanying drawings.

### DETAILED DESCRIPTION

Reference will now be made in detail to example embodiments, examples of which are illustrated in the accompanying drawings. The detailed description provided below in connection with the appended drawings is intended as a description of the present examples and is not intended to represent the only forms in which the present example may be constructed or utilized. The description sets forth the functions of the example and the sequence of operations for constructing and operating the example. However, the same or equivalent functions and sequences may be accomplished by different examples.

The optimal excitation of multi-transmitter wireless power transfer systems is dependent on the position of the receiver. Typically, all the transmitters are connected to the same power source and excited simultaneously by the power source. However, such simultaneous and homogeneous activation may not be optimal because the power contribution from each Tx to the Rx depends on the mutual coupling between each Tx-Rx pair. The mutual coupling, measured as mutual inductance, is the interaction of one coil's magnetic field on another coil as it induces a voltage in the adjacent coil. The mutual coupling may be estimated, for example, using a position sensor or with a wireless communication link configured between Tx and Rx to provide measurements from the Rx side to the Tx side. However, these solutions increase cost and complexity of the wireless power transfer system.

An objective of the presented description is to provide a method to determine the position of the receiver based on measurements obtained only from the transmitter coils. The position of the receiver may be indicated by a profile of current distribution coefficients (CDCs). The CDCs may be determined based on induced voltages at the transmitter coils and associated mutual inductance ratios between the transmitter coils and the receiver. The current distribution may be optimal for excitation such that it depends on the position of the receiver with respect to the transmitter coils.

According to an example embodiment, excitation of individual transmitters may be determined without using any measurements at the receiver side. In an embodiment, coil currents in each transmitter coil may be determined based on the mutual coupling, i.e. mutual inductances, between the Tx-Rx coil pairs using voltage measurements from the Tx side. The transmitter coils closer to the receiver may be supplied with higher currents compared to the ones farther away based on the determined current distribution. Hence, increased output power and efficiency may be achieved as transmitter coils with strong coupling to the receiver transfer more power than the transmitter coils with weak coupling.

According to an example embodiment, each transmitter coil is arranged to be substantially uncoupled from adjacent transmitter coils. The transmitter coils are uncoupled from each other such that there is no substantial mutual inductance between them. In this configuration, the optimal current in each transmitter coil is a function of the mutual inductances of each Tx-Rx coil pair. In an embodiment, the multiple transmitters may be positioned in a planar, pad-like arrangement to charge, for example, electric vehicles, mobile devices, or medical implants. In an embodiment, the transmitter coils may be positioned to form any shape as long as they are in a substantially uncoupled arrangement. The uncoupling arrangement may further enable, that only proper receivers are detected for wireless power transfer instead of, for example, nearby metal objects.

In an embodiment, individual Tx coils are activated one-by-one and induced voltage across the inactive Tx coils may be measured. The Tx coils may be activated one-by-one such that only one Tx is activated at a time. Tx coil may be activated when it is connected to a power source. The inactive Tx coils may be open and there is no current through them. When a Rx is absent, the induced voltages across inactive Tx coils may be close to zero due to the minimal coupling between the Tx coils. However, when a receiver is present, the inactive Tx coils may be coupled through the Rx, inducing voltage across them. Based on the induced voltages, mutual inductance ratios may be determined. The mutual inductance ratios may be used to determine the current distribution for the transmitters. This enables, that output power for wireless power transfer from the transmitter coils to the receiver may be optimized without any measurements required from the receiver.

An example embodiment may enable a simple and effective implementation for determining the coil currents of transmitter coils in a device configured for multi-transmitter wireless power transfer. No additional sensing circuitry is required to detect presence of the receiver. Further, no communication with the receiver or additional detection coils is needed. The measurement procedure for determining mutual inductance ratios of the Tx-Rx pairs is simple such that no substantial computational power is required. This enables, that effectiveness of the wireless power transfer by the device may be increased.

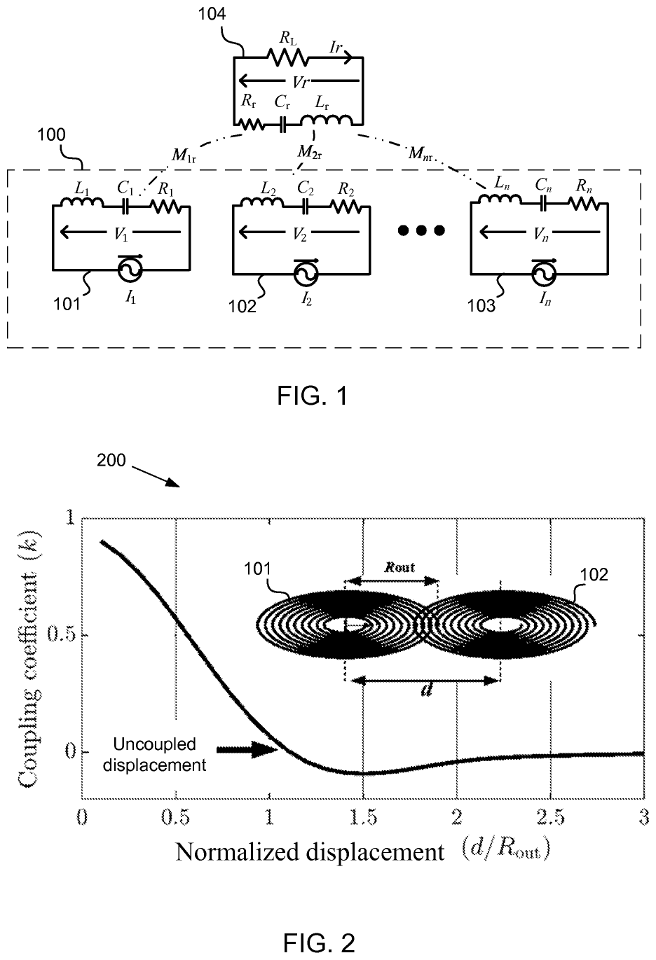

FIG. 1 illustrates an example of an equivalent circuit of a device 100 comprising multiple transmitter coils 101, 102, 103 for wireless power transfer to a receiver 104 according to an embodiment. The device 100 may comprise at least three transmitter coils 101, 102, 103. The device 100 may be configured to charge a load R_{L} of one or more receivers 104. The receiver 104 may be configured to charge, for example, a battery of a mobile phone, a laptop computer, a wearable device, or any other mobile device. In an embodiment, the receiver 104 may be configured to charge batteries of an electric vehicle.

Each of the transmitter coils 101, 102, 103 may be powered simultaneously or individually by a power source, which is illustrated by current sources I₁, I₂, Iₙ. The equation of the whole system comprising the device 100 and the receiver 104 may be written as

[V]_{(n+1)×1}=[Z]_{(n+1)×(n+1)}[I]_{(n+1)×1}, (1)

where

[V]_{(n+1)×1}=[V₁,V₂, . . . ,Vₙ,Vᵣ]^{T},

[I]_{(n+1)×1}=[I₁,I₂, . . . ,Iₙ,Iᵣ]^{T},

are the voltages V and currents I at the transmitter coils 101, 102, 103 (with subscripts 1, 2, . . . . n) and receivers 104 (with subscript r). The voltages and currents of the transmitter coils 101, 102, 103 and the receivers 104 are related by the impedance matrix [Z]_{(n+1)×(n+1)}, in which the elements have values:

$${[ Z ]}_{sl} = \{ \begin{matrix}{Z}_{s} = {R}_{s} + {jX}_{s} , & s = l \neq n + 1 \\ {Z}_{s} = {R}_{r} + {jX}_{r} , & s = l = n + 1 \\ {Z}_{sl} = {Z}_{ls} = j ω {M}_{sl} , & s \neq l ,\end{matrix} \qquad ( 2 )$$

wherein the diagonal terms Zₛ are the self-impedance of the n Txs 101, 102, 103 (s=1, 2, . . . , n) and Rx 104 (s=n+1 or r) coils, which comprise parasitic resistance Rₛ and reactance Xₛ=ωLₛ−1/ωCₛ (Lₛ and Cₛ represent the inductance L₁, L₂, . . . , Lₙ, Lᵣ and capacitance C₁, C₂, . . . , Cₙ, Cᵣ of each coil, respectively). The off-diagonal terms representing the impedance from mutual couplings, however, are nearly purely reactive, and Mₛₗ (=Mₗₛ) is the mutual inductance between the s^{th} coil and l^{th} coil. ω is the operational angular frequency of the power source I₁, I₂, . . . , Iₙ.

By expanding the last row of the equation (1) and assuming that Vᵣ=−IᵣR_{L}, wherein R_{L} is the equivalent load resistance at Rx, Rx current Iᵣ may be obtained:

$${I}_{r} = ( {\sum }_{s = 1}^{n} j ω {M}_{st} {I}_{s} ) / ( {R}_{r} + {R}_{L} + {jX}_{r} ) . \qquad ( 3 )$$

The output power at the receiver 104 is

Pₒᵤₜ=|Iᵣ|²R_{L}, (4)

and the efficiency of the wireless link equals

$$η = \frac{{P}_{out}}{{P}_{in}} = \frac{{\vert {I}_{r} \vert}^{2} {R}_{L}}{{\sum }_{s = 1}^{n} {\vert {I}_{s} \vert}^{2} {R}_{s} + {\vert {I}_{r} \vert}^{2} ( {R}_{r} + {R}_{L} )} . \qquad ( 5 )$$

To increase the output power and efficiency, the magnitude of the Rx current Iᵣ may be maximized while reducing the parasitic losses within the coils. Based on equation (3), operation at the resonance frequency of the Rx is recommended, so that Xᵣ=0. Also, summation of the MₛᵣIₛ product may be maximized. On the other hand, based on equation (5), the summation of the |Iₛ|²Rₛ product may be minimized. Because the parasitic resistances Rₛ (R₁, R₂, . . . , Rₙ, Rᵣ) are approximately the same for all the coils, currents Iₛ in each Tx coil 101, 102, 103 may be optimized according to the mutual inductances M₁ᵣ, M₂ᵣ, . . . , Mₙᵣ between each Tx-Rx pair.

The currents in each Tx coil may be represented as

$$[ \begin{matrix}{I}_{1} \\ {I}_{2} \\ ⋮ \\ {I}_{n}\end{matrix} ] = [ \begin{matrix}{α}_{1} \\ {α}_{2} \\ ⋮ \\ {α}_{n}\end{matrix} ] {I}_{0} , \qquad ( 6 )$$

where α_{1, 2, . . . , n} are CDCs that determine the currents in each Tx coil 101, 102, 103 relative to a nominal current I₀.

The nominal current I₀ may be determined depending on the power requirement. For example, when the Rx is positioned closest to the first transmitter 101, the transmitter current I₁ may be determined to have the largest value, and if the Rx moves closer to the second transmitter 102, the transmitter current I₂ may be determined to have the largest value. For efficient power transfer from multi-Tx coils 101, 102, 103 to the Rx 104, the CDCs may be derived as

$${α}_{s} = {M}_{sr} / \sqrt{{\sum }_{l = 1}^{n} {M}_{lr}^{2}} = 1 / \sqrt{{\sum }_{l = 1}^{n} {( \frac{{M}_{lr}}{{M}_{sr}} )}^{2}} . \qquad ( 7 )$$

Based on the equation (7), the mutual inductance ratios between each Tx-Rx pair is the decisive parameter for determining the efficient current distribution for the wireless power transfer. In equation (7), Mₗᵣ represents the mutual inductance between the l^{th} transmitter coil and the receiver 104, and Mₛᵣ the mutual inductance between the s^{th} transmitter coil and the receiver 104. The estimation of the mutual inductance ratios

$$\frac{{M}_{lr}}{{M}_{sr}}$$

between different transmitter coils 101, 102, 103 and the receiver 104 may be enough for achieving the efficient power transfer from the multi-Tx power transfer device 100. Hence, absolute values of the mutual inductances M₁ᵣ, M₂ᵣ, . . . , Mₙᵣ may not be required. Further, the mutual inductance ratios may be determined without requiring any current or voltage measurements from the receiving side.

In an embodiment, the at least three Tx coils 101, 102, 103 may be arranged so that all the Tx coils 101, 102, 103 are substantially uncoupled. When the adjacent Tx coils 101, 102, 103 are substantially uncoupled, the interactions between Tx coils 101, 102, 103 may be kept minimal in the absence of receivers 104. With the substantially uncoupled Tx coils 101, 102, 103, the impedance matrix may be simplified to

$${[ Z ]}_{sl} = [ \begin{matrix}{Z}_{1} & 0 & … & 0 & j ω {M}_{1 r} \\ 0 & {Z}_{2} & … & 0 & j ω {M}_{2 r} \\ ⋮ & ⋮ & ⋱ & ⋮ & ⋮ \\ 0 & 0 & … & {Z}_{n} & j ω {M}_{nr} \\ j ω {M}_{1 r} & j ω {M}_{2 r} & … & j ω {M}_{nr} & {Z}_{r}\end{matrix} ] . \qquad ( 8 )$$

FIG. 2 shows an example of positioning of two adjacent transmitter coils 101, 102 such that they may be uncoupled. The diagram 200 in FIG. 2 illustrates coupling coefficient k variation between the two transmitter coils 101, 102, which may be planar circular spiral coils, with respect to a normalized displacement d/Rₒᵤₜ between them. Rₒᵤₜ is the radius of the transmitter coil 101, 102 and d is the lateral displacement measured between centers of the transmitter coils 101, 102. Generally, the coupling coefficient k represents the amount of inductive coupling that exists between the two coils expressed as a fractional number between 0 and 1, where 0 indicates zero or no inductive coupling, and 1 indicates full or maximum inductive coupling. The circular planar transmitter coils 101, 102 may have some overlap between them, and the lateral displacement d between the coils may be selected such that the coupling coefficient k is ideally zero (marked as uncoupled displacement). However, in reality, the decoupling may be approximate such that the coupling of the Tx coils 101, 102, 103 is negligibly small compared to coupling between the transmitter coils 101, 102, 103 to the Rx 104. This enables that unwanted couplings between Tx coils may be avoided when Rx is absent. In addition, with the displacement configuration, the coupling between non-adjacent coils may also be negligibly small due to the large distance. Each Tx coil 101, 102, 103 may be positioned such that the distances between adjacent Tx coils 101, 102, 103 equals to the zero-coupling displacement d/Rₒᵤₜ, where coupling coefficient k between the adjacent Tx coils 101, 102, 103 is approximately zero. The required displacement may vary based on the shape and geometry of the transmitter coils.

In an embodiment, one of the transmitter coils 101, 102, 103 is activated by connecting it to the power source while the other transmitter coils remain inactive. When the single Tx coil is activated, there may not be any induced voltage across any other Tx coil if there is no Rx coil, because there may not be any coupling between the transmitter coils. When the Tx coil is activated, there may be some nominal current in the Tx coil. The nominal current may be relatively small, and it does not need to be the required current for charging the load. On the other hand, when the Rx is present, there may be induced voltages across the inactive Tx coils due to couplings Mₛᵣ from the Rx coil. The current of the active Tx coil may induce voltage and current to the Rx, and the induced current in Rx may further induce voltage to the inactive Tx coils having coupling with the Rx. By analyzing the induced voltages, the mutual inductance ratios may be determined. For example, when the first transmitter coil 101 is activated, the Rx 104 may induce voltage to the second transmitter coil 102, which induced voltage is related to the mutual inductances M₁ᵣ, M₂ᵣ. Each transmitter coil 101, 102, 103 may be activated one at a time to measure the induced voltage across each transmitter coil 101, 102, 103 to determine the mutual inductance ratios between each transmitter-receiver coil pair.

In an example embodiment, m^{th} Tx coil is connected to a power source and there is an Rx present. As all the other Tx coils (marked by s) are open, there may be no current through them, i.e., Iₛ=0. However, there may be voltage induced in the inactivated Tx coils and the voltage measured across the s^{th} Tx coil, Vₛₘ, may be obtained from equations (1) and (8) as

Vₛₘ=jωMₛᵣIᵣₘ, (9)

where Iᵣₘ is the Rx current when the m^{th} Tx coil is active. When the operation frequency is identical to the resonance frequency of the Rx, Iᵣₘ may be evaluated as

$${I}_{rm} = \frac{J ω {M}_{sr} {I}_{m}}{{R}_{r} + {R}_{L}} , \qquad ( 10 )$$

where Iₘ is the current in the m^{th} Tx coil. By measuring the induced voltages Vₛₘ, Vₗₘ across two different Tx coils, e.g., s^{th} and l^{th} Tx coils, when the m^{th} Tx coil is active (subscripts s, l, and m are different), the mutual inductance ratio may be determined from

$$\frac{{M}_{sr}}{{M}_{lr}} = \frac{{V}_{sm}}{{V}_{lm}} . \qquad ( 11 )$$

The measurement procedure may be repeated by connecting the power source to each Tx coil of the device for wireless power transfer one after another while all other transmitter coils are not powered. Hence, all mutual inductance ratios associated with the transmitter coils and the Rx may be determined. In an embodiment, the same mutual inductance ratio (associated to the same transmitter-receiver coil pair) may be determined using different induced voltage measurements when different transmitter coils are activated for verification purposes. Once the mutual inductance ratios are determined, the optimal or preferred CDC may be calculated using the equation (7). Further, based on equations (9) and (10), the induced voltage Vₛₘ is proportional to the product of the mutual inductance between the m^{th} Tx coil and the Rx and the s^{th} Tx coil and Rx, i.e. to MₘᵣMₛᵣ. Therefore, by analyzing the profile of the mutual inductance ratios and the products, the approximate position of Rx may be estimated. The preferred current distribution may be determined for different receiver positions based on the mutual inductance ratios as the change in orientation changes also the mutual inductances.

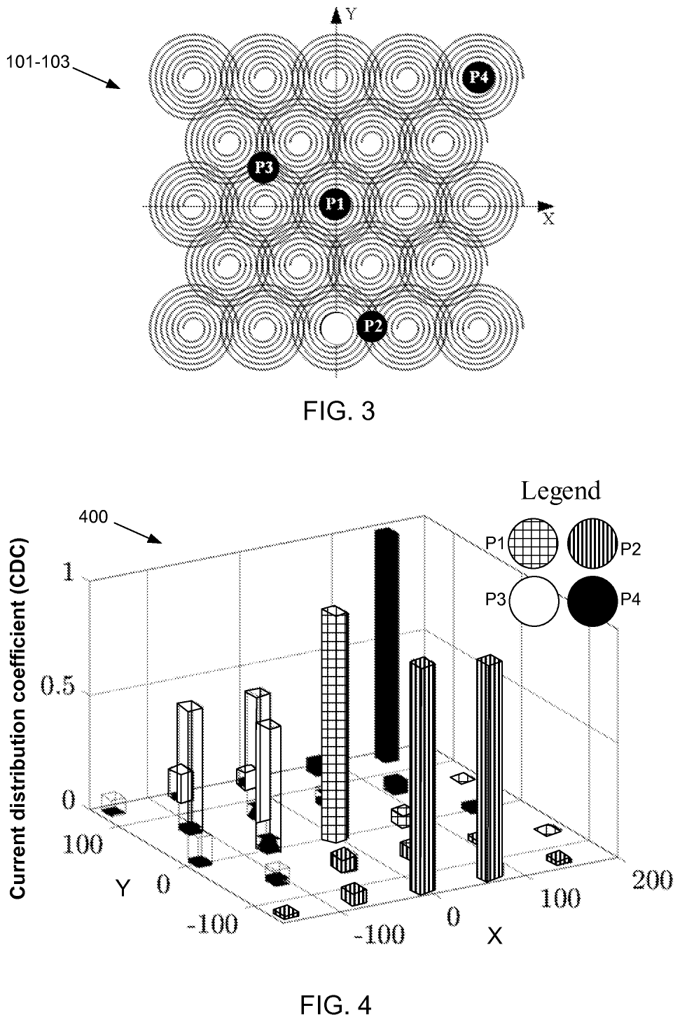

FIG. 3 illustrates an example arrangement of disc-shaped transmitter coils for multi-transmitter wireless power transfer with four possible locations P1, P2, P3, P4 of a receiver. The arrangement may comprise at least three transmitter coils 101, 102, 103. The CDC of each transmitter coil 101, 102, 103 for the four Rx positions P1, P2, P3, P4 are illustrated in a diagram 400 in FIG. 4. The CDC of the transmitter coils 101, 102, 103 may be determined based on induced voltage measurements as described above. The vertical (along z-direction) wireless power transfer distance between the transmitter coils 101, 102, 103 and the receiver may be, for example, 15 mm. In principle, the proposed Rx positioning approach may be used with any transfer distance. However, measurement sensitivity may be affected when the transfer distance is very large due to the very small mutual inductances.

Based on FIGS. 3 and 4, the Rx position may be easily and reliably estimated from the CDC profile. For instance, the receiver is aligned to a single Tx in positions P1 and P4. In FIG. 4, the positions P1 and P4 of the receiver are indicated by a peak CDC value for the respective Tx coils, while the currents in other Tx coils remain relatively small. Similarly, position P2 of the receiver is in between two Tx coils, and magnitude of the CDC values of the two Tx coils are substantially higher than the CDC values of the other Tx coils. Distances from position P3 of the receiver to the three closest Tx coils are the same, which is indicated in the diagram 400 in FIG. 4 by the equally increased CDC values for the respective Tx coils near the Rx. Again, CDC values for the other Tx coils locating farther away from the position P3 of the Rx are relatively small compared to the transmitters closest to the Rx.

In case of multiple Rxs, the position of each Rx may be determined as long as the Rxs are not too close to each other. In a real scenario, the receivers are usually kept at some distance from each other to avoid unwanted coupling between the receivers. In an embodiment, the profile of the mutual inductance ratios and respective CDC values may be analyzed to detect multiple Rxs. In an embodiment, the measurement procedure may be performed at predetermined intervals. Hence, a new receiver may be detected as well as a change in the position of a receiver.

In an embodiment, the voltage measurements may be used for sensing application to detect resonant and magnetically coupled objects, such as receivers, from other objects. In wireless power transfer applications, differentiating a metal object from a receiver may be a problem. When a metal object, such as a coin or a key, is close to the transmitter coils, current may be induced to the metal object from the activated transmitter coil. As a result, the metal object may heat and dissipate the energy. However, as the metal object may not be resonant, there is very small circulating current in the metal object, and therefore, no coupling with the inactive coils. Therefore, there is no induced voltage across the inactive coils and the metal object is not falsely detected as a receiver. In an embodiment, measurements from the transmitter coils may be used for metal detection. When there is a metal object near an active transmitter coil, there may be an increase in power from the active Tx coil drawn by the metal object. Hence, power measurements obtained from the transmitter coils may be used to detect a presence of a metal object.

In an embodiment, the voltage measurements may be performed around an area where higher mutual inductance ratios are detected. The higher mutual inductance ratios may indicate that receiver is somewhere close. After each voltage measurement, the mutual inductance ratio may be compared to the previously determined mutual inductance ratios, if any. The comparison may be performed for measurements performed during the current interval. If increase in the mutual inductance ratio is detected, one of the transmitter coils adjacent to the transmitter coil associated with the higher mutual inductance ratio may be selected as the next transmitter coil to be activated. On the other hand, the transmitter coils near a transmitter coil associated to a decreased mutual inductance ratio may not need to be activated, depending on the application. Hence, each transmitter coil may not need to be activated once the approximate location of the receiver is already detected.

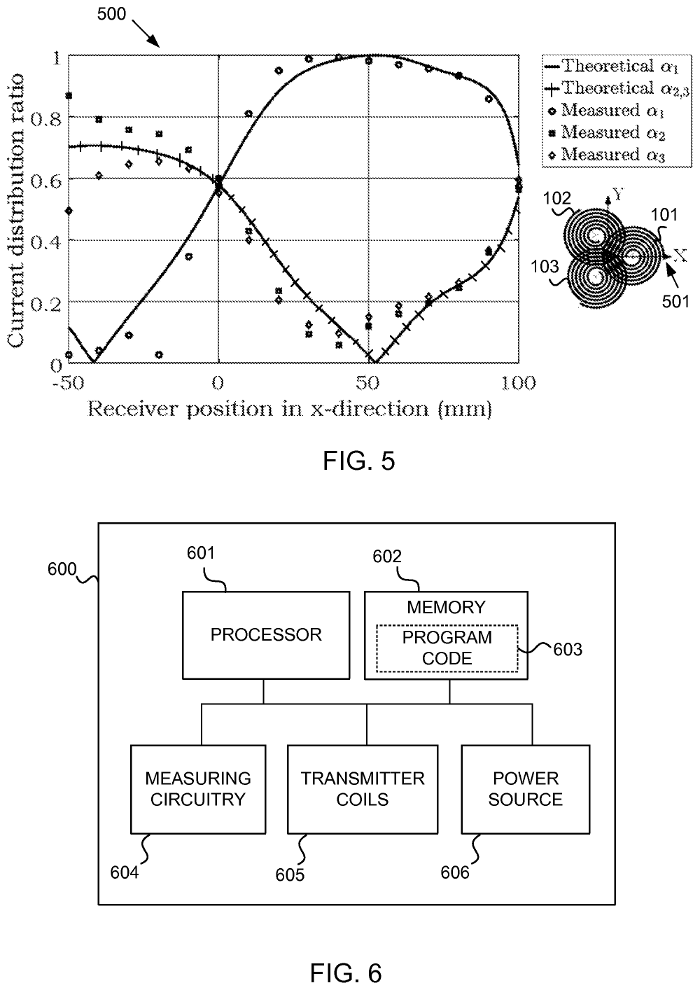

FIG. 5 illustrates an example diagram 500 of theoretical and experimental results of CDCs with respect to three transmitter coils 101, 102, 103 of a prototype device, based on positions of a receiver according to an embodiment. The measurements are performed with a prototype of a multi-transmitter wireless power transfer device comprising three disc-shaped transmitter coils 101, 102, 103 configured to transfer power to a single receiver coil. Example parameters of the transmitter and receiver coils and the prototype setup are illustrated in Table 1:

TABLE 1
The parameters of the prototype

| | |
|---|---|
| Parameter | Value |

Coil Design

| | | |
|---|---|---|
| Outer diameter | 100 | mm |
| Turns separation | 4 | mm |

| | |
|---|---|
| Number of turns | 10 |

Wireless Power Transfer Setup

| | | |
|---|---|---|
| Coil inductance | 6 | μH |
| Load | 1 | Ω |
| Operating frequency | 3.58 | MHz |
| Transfer distance | 15 | mm |

The Tx coils 101, 102, 103 are arranged in planar such that they are slightly overlapped and there is approximately zero coupling coefficient between them. Arrangement of the Tx coils 101, 102, 103 is illustrated in an inset of FIG. 5. The position of the Rx coil is varied along the x-axis 501 of the Tx coil arrangement. For a given Rx position, each transmitter coil 101, 102, 103 is connected to an RF power amplifier separately and the induced voltages across the other two Tx coils are measured using an oscilloscope. Next, the measured voltages are used to estimate the mutual inductance ratios using the equation (11). Finally, the optimal CDC, based on measured values α_{1,2,3}, are evaluated using the equation (7). The measured results (dots) of current distribution are compared with theoretical calculations (lines) in the diagram 500 in FIG. 5. This demonstrates that the measurement results substantially correspond with the theoretical calculations.

FIG. 6 illustrates an example embodiment of a device 600 configured to practice one or more example embodiments. The device 600 may comprise at least one processor 601. The at least one processor may comprise, for example, one or more of various processing devices, such as for example a co-processor, a microprocessor, a digital controller, a digital signal processor (DSP), a processing circuitry with or without an accompanying DSP, or various other processing devices including integrated circuits such as, for example, an application specific integrated circuit (ASIC), a field programmable gate array (FPGA), a microcontroller unit (MCU), a hardware accelerator, a special-purpose computer chip, or the like.

The device 600 may further comprise at least one memory 602. The memory 602 may be configured to store, for example, computer program code or the like, for example operating system software and application software. The memory 602 may comprise one or more volatile memory devices, one or more non-volatile memory devices, and/or a combination thereof. For example, the memory may be embodied as magnetic storage devices (such as hard disk drives, magnetic tapes, etc.), optical magnetic storage devices, or semiconductor memories (such as mask ROM, PROM (programmable ROM), EPROM (erasable PROM), flash ROM, RAM (random access memory), etc.).

The device 600 comprises at least three transmitter coils 605. The number and configuration of the transmitter coils 605 may depend on application. The transmitter coils 605 may comprise at least one of the transmitter coils 101, 102, 103. The device 600 may further comprise a measuring circuitry 604 coupled to the transmitter coils 605. The measuring circuitry 604 may be configured to measure voltage across single transmitter coils 605. The measuring circuitry 604 may comprise, for example, a voltage transducer, a voltage measuring unit, a voltage sensor, or the like. The device 600 may further comprise a power source 606 coupled to each transmitter coil 605. The power source 606 may be configured to provide power to each transmitter coil 605 simultaneously and/or individually.

When the device 600 is configured to implement some functionality, some component and/or components of the device 600, such as for example the at least one processor 601 and/or the memory 602, may be configured to implement this functionality. Furthermore, when the at least one processor 601 is configured to implement some functionality, this functionality may be implemented using program code 603 comprised, for example, in the memory 602.

In an embodiment, the memory 602 may comprise program code 603 which, when executed by the processor 601, causes the device 600 to activate, by the power source 606, the transmitter coils 605 one at a time with a relatively small current. When one of the transmitter coils 605 is activated, the measuring circuitry 604 may be configured to measure induced voltages across at least two transmitter coils 605 adjacent to the activated transmitter coil. Each voltage measurement is performed across single transmitter coil. The measurement results may be processed by the processor 601 to obtain mutual inductance ratios of possible transmitter-receiver pairs. The device 600 may be configured to analyze the mutual inductance ratios to determine CDCs indicative of position of the receiver. In an embodiment, the device 600 may be configured to initiate wireless power transfer to the receiver by powering the transmitter coils 605 by the power source 606 based on the determined CDCs.

The functionality described herein may be performed, at least in part, by one or more computer program product components such as software components. According to an embodiment, the device 600 comprises a processor or processor circuitry, such as for example a microcontroller, configured by the program code when executed to execute the embodiments of the operations and functionality described. Alternatively, or in addition, the functionality described herein can be performed, at least in part, by one or more hardware logic components. For example, and without limitation, illustrative types of hardware logic components that can be used include Field-programmable Gate Arrays (FPGAs), application-specific Integrated Circuits (ASICs), application-specific Standard Products (ASSPs), System-on-a-chip systems (SOCs), Complex Programmable Logic Devices (CPLDs), Graphics Processing Units (CPUs).

The device 600 comprises means for performing at least one method described herein. In one example, the means comprises the at least one processor 601, the at least one memory 602 including program code 603 configured to, when executed by the at least one processor 601, cause the device 600 to perform the method.

The device 600 may comprise, for example, a wireless charger device for cordless or mobile appliances. The device 600 may be, for example, a charging pad, a charging mat or a charging station. Although the device 600 is illustrated as a single device it is appreciated that, wherever applicable, functions of the device 600 may be distributed to a plurality of devices.

In an embodiment, the device 600 may be integrated to a furniture, such as a table. In an embodiment, the device 600 may be installed to an infrastructure, such as to a roadway, a floor or a parking lot, for example, for charging of electric cars, electric wheelchairs, electric scooters, warehouse robots, or the like. In an embodiment, the device 600 may be installed in a warehouse floor for charging, for example, warehouse robots. This may enable that the warehouse robot gets always charged when it comes to the charging area comprising the transmitter coils 605. This may even allow the warehouse robots to work 24/7 without needing to separately charge them in a specified location, such as at a charging dock.

As long as each transmitter coil 605 is substantially uncoupled from the other transmitter coils 605, the transmitter coils 605 may be arranged to form any shape. In an embodiment, the transmitter coils 605 may be arranged in a ball-shape. In an embodiment, the transmitter coils may be arranged in a container shape. In an embodiment, the transmitter coils 605 may be positioned in a planar arrangement. In an embodiment, when the transmitter coils 605 are arranged, for example, in the ball-shape or the container-shape, the device 600 may be configured to charge an object placed inside the transmitter coil 605 arrangement.

In an embodiment, the device 600 may be configured to repeat the measurement procedure at predetermined intervals. The device may be configured to charge, for example, a laptop, which may take 20 W power. The transmitter coils may be activated by the device one at a time with a power value higher than noise levels, for example, 0.5 W. The activation current may be small, and it does not need to be the current required for charging the laptop. The measurements may be repeated at predetermined intervals, for example, every 2 s, every 10 s, every 30 s, or every minute, by injecting the small current throughout the whole transmitter coil arrangement of the device 600 one by one. If no receiver is detected because there are no induced voltages, the device 600 may enter a sleep mode. The sleep mode may be a low power mode where power for unneeded components are shut down. If receiver is detected, it may be charged by the device 600 based on the determined CDC for the transmitter coils 605.

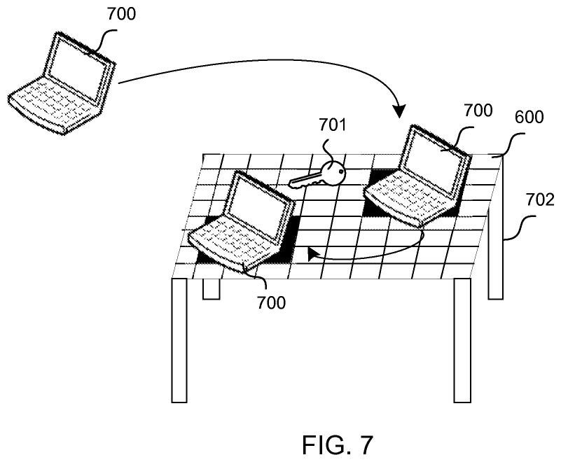

In an embodiment, the device 600 may be configured to charge one or more mobile devices. In an embodiment, the device may be integrated to a furniture, such as a tabletop of a table 702 as illustrated in FIG. 7. The transmitter coils of the device 600 are illustrated as a grid, and they may cover the surface of the tabletop. When there is no mobile device 700 detected near the device 600, the device 600 may be in the sleep or a stand-by mode for the predetermined interval. The mobile device 700 may be placed anywhere near the transmitter coils to initiate charging. The mobile device 700 may be efficiently charged by the device 600 as the location of receiver of the mobile device 700 may be reliably detected. The device 600 may also detect when one or more additional mobile devices are placed near it for charging. The device 600 may determine higher currents for the transmitter coils nearest to the detected receivers (illustrated with black-colored grids) based on the CDC to provide efficient power output for each one. Even if one or more mobile device moves from the initially detected location to a different location within a range of the transmitter coils, the current distribution may be changed by the device 600 accordingly. The current distribution may be changed, for example, at the predetermined intervals or continuously based on detecting the respective receivers at the new locations from changed CDC profile. The device 600 may provide mobility for the receivers configured to receive wireless power transfer while effective power transfer is ensured.

When the device is integrated to a furniture, the furniture may be used to safely store other objects, while mobile devices are being charged. The other objects may comprise for example metal objects 701, such as keys or coins. The device 600 may be able to detect only actual receivers for charging from the induced voltages at the transmitter coils, and therefore power supply is not unnecessary initiated for other objects nearby the transmitter coils. Therefore, unnecessary power usage or heating of metal objects may be avoided. In an embodiment, the device 600 may be configured to detect metal objects 701. The device 600 may obtain power measurements associated with the active transmitter coil. The power measurements may be obtained based on power supply to the active transmitter coil and/or measured voltage across the active transmitter coil. In response to an increase in the power from the active transmitter coil, while there are no induced voltages across the adjacent transmitter coils, the device 600 may determine that there is a metal object 701 near the active transmitter coil. When the metal object 701 is detected and there are no receivers, the device 600 may enter the sleep mode. In an embodiment, the device 600 may notify a user about the detected metal object, for example, via a communication interface configured to transmit and/or receive information to/from other devices. The device 600 may be able to warn the user about the metal object, for example, so that heating of the metal object may be avoided when there is a receiver being charged near the metal object 701.

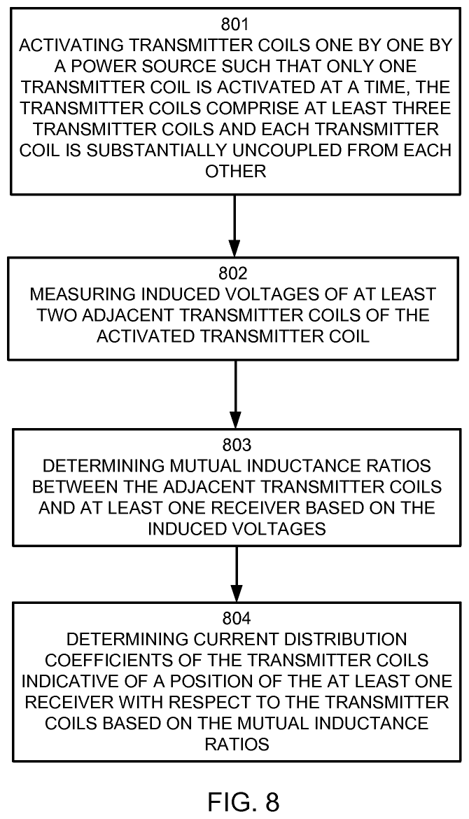

FIG. 8 illustrates an example of a method for determining a position of a receiver based on measurements from transmitter coils, according to an example embodiment.

At 801, the method may comprise activating transmitter coils one by one by a power source such that only one transmitter coil is activated at a time. The transmitter coils may comprise at least three transmitter coils, wherein each of the transmitter coils are substantially uncoupled from each other. The transmitter coils may be arranged in any shape, such as in planar, in a ball-shape or in a container-shape as long as they are positioned such that there is a displacement between them resulting approximately zero coupling coefficient between all transmitter coils.

At 802, the method may comprise measuring induced voltages of at least two adjacent transmitter coils of the activated transmitter coil. After the induced voltages of the adjacent transmitter coils are measured, the activated transmitter coil may be inactivated. Thereafter, next transmitter coil may be activated, while all the other transmitter coils remain inactive, for measuring induced voltages of transmitter coils adjacent to the currently active transmitter coil.

At 803, the method may comprise determining mutual inductance ratio between the adjacent transmitter coils and at least one receiver based on the induced voltages. In an embodiment, the method may comprise comparing the mutual inductance ratios after each induced voltage measurements to detect transmitter coils associated with higher mutual inductance ratios; and activating next only the transmitter coils adjacent to the transmitter coils associated with the higher mutual inductance ratios. In an embodiment, the transmitter coils may be activated one at a time at a predetermined interval such that the measurements may be repeated at certain intervals. In an embodiment, the measurement may indicate that there is no induced voltage due to the absence of any receiver, and hence a sleep mode of the system may be initiated.

At 804, the method may comprise determining CDCs of the transmitter coils indicative of a position of the at least one receiver with respect to the transmitter coils based on the mutual inductance ratios. In an embodiment, the method may comprise initiating power supply to the transmitter coils according to the determined CDCs.

Further features of the method(s) directly result for example from functionalities of the device for multi-transmitter power transfer described throughout the specification and in the appended claims and are therefore not repeated here. Different variations of the method(s) may be also applied, as described in connection with the various example embodiments.

A device may be configured to perform or cause performance of any aspect of the method(s) described herein. Further, a computer program may comprise instructions for causing, when executed, a device to perform any aspect of the method(s) described herein. Further, a device may comprise means for performing any aspect of the method(s) described herein.

According to an example embodiment, the means comprises at least one processor, and memory including program code, the at least one processor, and program code configured to, when executed by the at least one processor, cause performance of any aspect of the method(s). Any range or device value given herein may be extended or altered without losing the effect sought. Also, any embodiment may be combined with another embodiment unless explicitly disallowed.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item may refer to one or more of those items.

The operations of the methods described herein may be carried out in any suitable order, or simultaneously where appropriate. Additionally, individual blocks may be deleted from any of the methods without departing from the scope of the subject matter described herein. Aspects of any of the embodiments described above may be combined with aspects of any of the other embodiments described to form further embodiments without losing the effect sought.

The term ‘comprising’ is used herein to mean including the method, blocks, or elements identified, but that such blocks or elements do not comprise an exclusive list and a method or device may contain additional blocks or elements.

Although subjects may be referred to as ‘first’ or ‘second’ subjects, this does not necessarily indicate any order or importance of the subjects. Instead, such attributes may be used solely for the purpose of making a difference between subjects.

It will be understood that the above description is given by way of example only and that various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from scope of this specification.

1-23. (canceled)

24. A device comprising:
at least three transmitter coils, the transmitter coils arranged such that all the transmitter coils are substantially uncoupled from each other;
a measuring circuitry configured to measure voltage across individual transmitter coils;
a power source configured to supply power to the transmitter coils independently or simultaneously;
a processor; and
a memory comprising program code which, when executed by the processor causes the device at least to:
activate transmitter coils one by one by the power source such that only one transmitter coil is activated at a time;
measure, by the measuring circuitry, induced voltages of at least two transmitter coils adjacent to the activated transmitter coil;
determine mutual inductance ratios between the adjacent transmitter coils and at least one receiver (104) based on the induced voltages; and
determine current distribution coefficients of the transmitter coils indicative of a position of the at least one receiver with respect to the transmitter coils based on the mutual inductance ratios.

25. The device of claim 24, wherein the device (100, 600) is further caused to initiate power supply to the transmitter coils according to the determined current distribution coefficients.

26. The device of claim 24, wherein the transmitter coils are positioned in a planar arrangement.

27. The device of claim 24, wherein the transmitter coils are positioned in a ball-shaped arrangement.

28. The device of claim 23, wherein the transmitter coils are positioned in a container-shaped arrangement.

29. The device of claim 23, wherein the transmitter coils are positioned such that a normalized displacement d/Rout between the adjacent transmitter coils results in an approximately zero coupling coefficient.

30. The device of claim 23 integrated to a furniture or infrastructure.

31. The device of claim 23, wherein the transmitter coils are activated one by one at a predetermined interval.

32. The device of claim 23 caused to:
compare the mutual inductance ratios after each induced voltage measurements to detect transmitter coils associated with higher mutual inductance ratios; and
activate next only the transmitter coils adjacent to the transmitter coils associated with the higher mutual inductance ratios.

33. The device of claim 23 caused to:
determine that there are no receivers nearby in response to the measurements indicating that no voltage is induced to any of the transmitter coils; and
enter into a sleep mode when there are no receivers nearby.

34. The device of claim 23 caused to:
measure power from the active transmitter coil;
detect an increase in the power from the active transmitter coil while there are no induced voltages across the adjacent transmitter coils; and
determine there is a metal object (701) near the active transmitter coil (101, 102, 103, 605).

35. The device of claim 34, further caused to enter into the sleep mode in response to detecting the metal object near the active transmitter coil while there are no receivers nearby.

36. The device of claim 34, further caused to notify a user about the metal object.

37. A method, comprising:
activating transmitter coils one by one by a power source such that only one transmitter coil is activated at a time, wherein the transmitter coils comprise at least three transmitter coils and all the transmitter coils are substantially uncoupled from each other;
measuring induced voltages of at least two adjacent transmitter coils of the activated transmitter coil;
determining mutual inductance ratios between the adjacent transmitter coils and at least one receiver based on the induced voltages; and
determining current distribution coefficients of the transmitter coils indicative of a position of the at least one receiver with respect to the transmitter coils based on the mutual inductance ratios.

38. The method of claim 37, comprising initiating power supply to the transmitter coils according to the determined current distribution coefficients.

39. The method of claim 37, wherein the transmitter coils are activated one by one at a predetermined interval.

40. The method of claim 37, further comprising:
comparing the mutual inductance ratios after each induced voltage measurements to detect transmitter coils associated with higher mutual inductance ratios; and
activating next only the transmitter coils adjacent to the transmitter coils associated with the higher mutual inductance ratios.

41. The method of claim 37, comprising:
determining that there are no receivers nearby in response to the measurements indicating that no voltage is induced to any of the transmitter coils (101, 102, 103, 605); and
entering into a sleep mode when there are no receivers nearby.

42. The method of claim 37, comprising:
measuring power from the active transmitter coil;
detecting an increase in the power from the active transmitter coil while there are no induced voltages across the adjacent transmitter coils; and
determining there is a metal object near the active transmitter coil.

43. The method of claim 42, further comprising entering into the sleep mode in response to detecting the metal object near the active transmitter coil while there are no receivers nearby.

44. The method of claim 42, further comprising notifying a user about the metal object.

45. A computer readable medium, comprising program code which, when executed by at least one processing unit, causes the at least one processing unit to perform the following steps:
activating transmitter coils one by one by a power source such that only one transmitter coil is activated at a time, wherein the transmitter coils comprise at least three transmitter coils and all the transmitter coils are substantially uncoupled from each other;
measuring induced voltages of at least two adjacent transmitter coils of the activated transmitter coil;
determining mutual inductance ratios between the adjacent transmitter coils and at least one receiver based on the induced voltages; and
determining current distribution coefficients of the transmitter coils indicative of a position of the at least one receiver with respect to the transmitter coils based on the mutual inductance ratios.
